(19) United States
(12) Patent Application Publication
(10) Pub. No.: US 2009/0082695 A1
(43) Pub. Date: Mar. 26, 2009

(54) METHODS, SYSTEMS AND APPARATUS RELATING TO COLPOSCOPIC-TYPE VIEWING EXTENSION DEVICES

(75) Inventor: Peter Whitehead, West Vancouver (CA)

Correspondence Address: GRAYBEAL JACKSON LLP, 155 - 108TH AVENUE NE, SUITE 350, BELLEVUE, WA, 98004-5973, US

(73) Assignee: LED Medical Diagnostics, Inc., White Rock (CA)

(21) Appl. No.: 12/146,387

(22) Filed: Jun. 25, 2008

Related U.S. Application Data
(60) Provisional application No. 60/937,259, filed on Jun. 25, 2007.

(51) Int. Cl. A61B 10/00

(52) U.S. Cl. 600/562; 600/572

(57) ABSTRACT

Handheld imaging and sampling probe systems, and related devices, kits, methods, etc., that may improve the general comfort of gynecological examinations and/or obtaining samples of cells from the vagina or cervix, and may also improve sample collection and thus possibly improve the sensitivity and specificity. The devices comprise an imaging and sample collection probe sized for easy and comfortable insertion into the vagina without requiring the use of a speculum comprising a proximally located handle and at least one relatively thin extension that extends distally from the handle. The probe further comprises at least one light emitter that emits illumination light toward the target tissue, at least one imaging system that detects and images examination light emanating from the target tissue, and a biopsy channel configured to transmit to the target tissue a retractable biopsy or surgical device able to retractably contact the target tissue, typically to obtain a sample of cells from the target tissue. The system can further comprise a disposable sleeve sized to substantially cover at least the part of the extension to be inserted into the vagina, the sleeve comprising at least one passage configured to securely align with the biopsy channel and sized to transmit the retractable sample collection device.

### CROSS-REFERENCE TO RELATED APPLICATIONS

The present application claims priority from U.S. provisional patent application No. Ser. No.: 60/937,259, filed Jun. 25, 2007, which is incorporated herein by reference in its entirety and for its teachings and disclosures.

### BACKGROUND

The Papanicolaou test (also called Pap smear, Pap test, cervical smear, or smear test) is a medical screening method designed, for example, to detect premalignant and malignant processes in the ectocervix, as well as infections and abnormalities in the endocervix and endometrium. The test remains a highly effective, widely used method for early detection of cervical cancer and pre-cancer.

The general sampling technique changed very little since its invention by Georgios Papanicolaou (1883-1962) to detect cyclic hormonal changes in vaginal cells in the early 20th century until the development of liquid based cell thinlayer technology. The physician or operator collecting a sample for the test inserts a speculum into the patient's vagina to obtain a cell sample from the cervix. Samples are collected from the outer opening or os of the cervix using a sampling device such as an Aylesbury spatula or (more frequently with the advent of liquid-based cytology) a plastic-fronded broom. The cells are placed on a glass slide and checked for abnormalities in the laboratory. Examples of improved systems, devices and the like for examining the cervix and, if desired, obtaining cell samples, are shown in commonly owned U.S. published patent application no. 2006/0241347, published Oct. 26, 2006.

Due to the anatomy of the vaginal and cervical areas, etc., improvements in consistent and reliable sampling when either the spatula or broom (or other sampling device) is used to obtain the samples such as the ectocervical sample, would be advantageous. The present systems and methods, etc., provide these and/or other advantages.

### SUMMARY

In one aspect, the devices, systems, procedures, etc., discussed herein may improve the general comfort of the procedure of gynecological examinations and/or obtaining samples of cells, and may improve the sample collection and thus possibly improve the sensitivity and specificity. The devices comprise an imaging probe sized for easy and comfortable insertion into the vagina, which the physician or the operator can insert into the vagina and up to the cervix typically without the use of a speculum. The about 1 cm diameter (for example) probe may be either direct viewing (i.e., permit the examiner to directly look at the tissue under examination) or have a CCD chip or other sensor (such as a light-pick-up device that transmits the image to a proximally-located imaging sensor) on the distal end that can transmit the image(s) to an ocular and/or monitor. The probe can have, e.g., the option of white light or blue light illumination, with the appropriate filters as desired for improving and highlighting areas of the cervix need to be sampled.

Prior to the insertion of the probes discussed herein into the patient, the probe is typically inserted into a disposable sleeve that can have a retractable sample collection device (SCD). The SCD may be, for example, either or a spatula or broom. The mechanism of retraction may, for example, either be a “ballpoint pen” spring approach or a second sleeve over the initial sleeve.

In one embodiment of use, the combined disposable sleeve and probe is inserted into the vaginal canal until just proximal to the cervix. The physician or operator can, for example, visually assess the cervix with white light and then with blue light. The contrast of blue light and if desired fluorescence induced by such blue light may improve the accuracy of where to take the sample. After visual examination, the physician or operator may take the sample using a sampler such as an Aylesbury spatula or bioptome provided with or through the probe, then retract the probe (including the sampler) from the vagina to ensure the sample does not become contaminated on exiting the vaginal canal. Once the probe has exited the patient, the sampler can be removed and the specimen can be processed.

These and other aspects, features and embodiments are set forth within this application, including the following Detailed Description and attached drawings. Unless expressly stated otherwise, all embodiments, aspects, features, etc., can be mixed and matched, combined and permuted in any desired manner. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 depicts a side plan view of one embodiment of a handheld imaging and sampling probe system as discussed herein.

FIG. 2 depicts a cutaway side view of another embodiment of a handheld imaging and sampling probe system as discussed herein wherein the probe comprises an ocular.

FIG. 3 depicts a side plan view of another embodiment of a handheld imaging and sampling probe system as discussed herein wherein the system comprises a biopsy channel and retractable sample collection device therein extending from the proximal end to the distal tip of the probe, and a sheath sized to cover the thin extension extending distally from the handle.

FIG. 4 depicts a top plan view of an embodiment of a handheld imaging and sampling probe comprising two biopsy channels and retractable sample collection devices.

FIG. 5 depicts a perspective view of a wireless embodiment of a handheld imaging and sampling probe system, the system further comprising a proximally located viewing screen.

FIG. 6 depicts a side plan view of a handle portion of a handheld imaging and sampling probe system.

FIG. 7 depicts a side plan view of an embodiment of a handheld imaging and sampling probe system having an in-line handle configuration and a flip-up viewing screen.

FIG. 8 depicts a side plan view of the handheld imaging and sampling probe system of FIG. 7 wherein the flip-up viewing screen is in the up position.

FIG. 9 depicts a perspective view of a handheld imaging and sampling probe system having an in-line handle configuration and a spring-loaded retractable sample collection device.

FIG. 10 depicts a perspective view of a spring-loaded retractable sample collection device.

FIG. 11 depicts a top plan view of a handheld imaging and sampling probe system having an in-line handle configuration and two spring-loaded retractable sample collection devices.

FIG. 12 depicts a side plan view of a handheld imaging and sampling probe system having an in-line handle configuration and a substantially distally located biopsy channel for a manually manipulated retractable sample collection device.

FIG. 13 depicts a cross-sectional view of a spring-loaded mechanism.

FIG. 14 depicts a perspective view of an exemplary arrangement for a distal tip of the handheld imaging and sampling probe system.

FIG. 15 depicts a perspective view of exemplary arrangements for a distal tip of the handheld imaging and sampling probe system.

FIG. 16 depicts an end plan view of exemplary arrangements for a distal tip of the handheld imaging and sampling probe system.

### DETAILED DESCRIPTION

The present handheld imaging and sampling probe systems, and related devices, kits, methods, etc., may improve the general comfort of gynecological examinations and/or obtaining samples of cells from the vagina or cervix or related structures, and may also improve sample collection and thus possibly improve the sensitivity and specificity. The devices comprise an imaging and sample collection probe sized for easy and comfortable insertion into the vagina without requiring the use of a speculum (a speculum can be used if desired but is not necessary). Thus, in one aspect the handheld probe comprises a proximally located handle and at least one relatively thin extension that extends distally from the handle. The handle is sized and configured to be maintained external to a patient and the extension is can reach the cervix or other desired internal structure of a patient. The probe further comprises at least one light emitter that emits illumination light toward the target tissue, at least one imaging system that detects and images examination light emanating from the target tissue, and at least one biopsy channel in at least the extension that is configured to transmit to the target tissue a retractable biopsy or surgical device able to retractably contact the target tissue, typically to obtain a sample of cells from the target tissue (as used herein, “retractable biopsy or surgical device” includes devices such as swabs that may not specifically be used to obtain samples or conduct surgeries). The system can further comprise a disposable sleeve sized to substantially cover at least the part of the extension to be inserted into the vagina, the sleeve comprising at least one passage configured to securely align with the biopsy channel and sized to transmit the retractable sample collection device.

Turning to some additional general discussion, the present methods, devices, etc., are directed in one aspect to colposcopic-type viewing devices configured such that a relatively thin extension extends from a handle and viewing screen/ocular. The extension is sized and configured to be easily insertable without discomfort into the vagina without using a speculum. The device represents an improvement, for example, over traditional speculums in that patient discomfort may be reduced while viewing options are enhanced for certain applications. For example, the colposcopic viewing device of the current structures, systems, etc., can be configured with a variety of optical elements, such as lenses, filters, interference gratings, etc., such that any desirable illumination/excitation light can be delivered to the target tissue (typically the cervix, but also possibly the os, or the vaginal walls, or otherwise as desired), including for example full white light, substantially blue light configured to excite one or more fluorophores under examination, infrared light, green light, or other selected light band(s) as desired. If desired, the examination light can include UV light but such is typically disfavored due to the potential for damage to the tissue under examination.

Similarly, any desired combination of optical elements, typically including lenses, filters, interference gratings, etc., can be provided to pass or filter out all or certain portions of the examination light removing from the target tissue, thereby enhancing the ability to detect desired target structures or diseases or conditions. For example, the returning light to be examined can substantially consist of reflectance light comprising substantially all of the white light or other desired illumination/excitation light transmitted to the target tissue, and/or can be substantially fluorescence or other form of light emanating from the target. Exemplary embodiments of suitable filters, lenses, gratings, etc., as well as combinations of such filters, lenses, etc., can be seen, for example, in U.S. Pat. No. 6,110,106 and U.S. PA 20050234526.

Typically, the colposcopic extension can provide either or both direct viewing where the eye of the examiner (typically a doctor, surgeon, nurse, etc.) is applied to an ocular or other suitable viewing device so that the light returning from the target tissue is transmitted through or along the colposcopic-type viewing extension device directly to the eye of the user. In addition, or instead, as desired, the colposcopic-type viewing extension device can also comprise one or more imaging sensors at the distal tip (or other location as desired, for example along the sides of the colposcopic-type viewing extension device to facilitate examination of the vaginal walls), which sensors are then operably coupled to a viewing screen disposed on or in the colposcopic-type viewing extension device. Typically, the viewing screen connected to the sensor is located such that the image transmitted from the sensor is directly transmitted to the viewing screen in a configuration such that movement of the distal tip of the colposcopic-type viewing extension device performs for the viewer as though the viewer were looking down a substantially straight tube and thus manipulation of the viewing sensor+viewing screen is intuitive and therefore easy to manage.

If desired, the image(s) obtained from the colposcopic-type viewing extension device can be transmitted, for example via wire, radio frequency, or otherwise as desired, from the colposcopic-type viewing extension device to a separate, stand-alone viewing device or system, such as a viewing screen, a computer, etc. If desired, one or more spectrometers can also be associated with the colposcopic-type viewing extension device.

Typically, the colposcopic-type viewing extension device can further comprise, for example in a side channel, one or more biopsy channels sized and configured to transmit a biopsy or surgical device such as a spatula, broom, punch biopsy device, surgical loop, etc., along the length of the colposcopic-type viewing extension device. Typically, such biopsy channel extends the complete length of the colposcopic-type viewing extension device, but, again, as desired, it may also have one or more side ports to facilitate activity with the vaginal wall or other structures as desired.

Typically, the colposcopic-type viewing extension device is of adequate length to reach the cervix and os of the patient but as desired, multiple lengths of colposcopic-type viewing extension device can be provided to more easily reach structures short of the cervix or os, or to accommodate differing vaginal tube lengths in different patients or otherwise as desired.

If desired, the colposcopic-type viewing extension device can further comprise one or more extendable elements sized and configured to penetrate the os. Such elements typically comprise one or more of imaging sensors, spectroscopic sensors (such spectroscopic sensors, as in other aspects of the structures and devices discussed herein, include spectrometers, spectrophotometers, spectroradiometers, spectrographs, etc.). The extendable elements can also include one or more biopsy or surgical devices as desired.

Typically, the viewing screen of the colposcopic-type viewing extension device displays the tissue under examination in true color, but if desired false color or other artificial renditions may be provided. In certain embodiments, specified detected wavelengths are enhanced relative to other wavelengths so that a viewer can more easily distinguish between various targets or the relative condition(s) of different areas within a target(s). In addition, if desired, the viewing device may comprise a snapshot camera. One advantage of such a configuration is that the illumination elements need not be as large nor powerful for a snapshot camera as for a continuous viewing device, which can be particularly advantageous for pediatric or other situations where diminished size of the colposcopic-type viewing extension device is desirable.

Typically, the colposcopic-type viewing extension device is substantially rigid from proximal end to distal end but articulatable sections or portions can be provided if desired. Additionally, the colposcopic-type viewing extension device is typically provided with one or more handles, either in line, or pistol-grip type or otherwise as desired. Further, the colposcopic-type viewing extension device can be hard-connected to external support devices such as light sources, computers, power sources, etc., or the colposcopic-type viewing extension device can be stand-alone and contain batteries, light sources, etc., wholly within a detached device. In the event that the colposcopic-type viewing extension device is detached (i.e., not physically tethered to a support device), the colposcopic-type viewing extension device can, if desired, include one or more wireless transmitters such as a radio transmitter to connect the device to one or more external support devices.

The colposcopic-type viewing extension device can be made of any desirable material, typically surgical grade plastics or metals. As desired, the colposcopic-type viewing extension device may further comprise one or more gradations or markings such that an additional viewing device, such as for example an MRI machine or PET machine or x-ray machine, can easily determine the location, relative position, size, etc., of both the colposcopic viewing device and, potentially, lesions, tumors, etc., that may be found within the patient as well as other structures that may be ordinarily found within a healthy patient.

In still further embodiments, the colposcopic-type viewing extension device can further comprise one or more channels that conduct a desired composition to the target, or extract a desired composition from the examination area. For example, the colposcopic-type viewing extension device can include a channel that can provide water, air, acetic acid or other liquid to the viewing area, or can provide suction to the viewing area.

If desired, the colposcopic-type viewing extension device can also be combined with other viewing devices such as sensors for sonograms, MRI, PET, etc.

In some basic embodiments, however, the colposcopic-type viewing extension device comprises a simple, substantially hollow tube through which or from which examination light is transmitted to the target tissue of the patient and from which examination light is returned from such target tissue of the patient (whether reflectance light, fluorescence light, phosphorescent light, etc.) to the viewer. The colposcopic-type viewing extension device is typically coupled with, as noted previously, either a direct viewing ocular and/or a direct viewing screen, which screen can be, for example, a ground glass screen coupled with a substantially empty channel by which direct viewing of the target tissue is attained, and/or an LCD or other type screen coupled to an appropriate sensor such as a CCD, CID, CMOS, etc., located typically at the distal tip of the colposcopic-type viewing extension device in substantially linear relation to the viewing screen so that, as noted earlier, manipulation of the colposcopic-type viewing extension device provides an intuitive change of the field of view. Also in such basic configuration, the colposcopic-type viewing extension device will typically include one or more open passages along the length of the colposcopic-type viewing extension device through which one or more biopsy or surgical tools can be passed to perform, for example, pap smears or other biopsies or minor surgical activities as desired. In certain embodiments, the colposcopic-type viewing extension devices consist essentially of the elements provided in the basic configuration and substantially no other additional or add-on features other than support devices ( light sources, computers) and stylistic elements.

If desired, the colposcopic-type viewing extension device can further comprise one or more swabs for obtaining cells from the target tissue. As desired, the swab may be operably connected to the proximal end of the colposcopic device such that the swab can be engaged by turning a handle, pushing a lever or otherwise such that the swab is operably engaged to a trigger-type handle whereby pulling or pushing the handle extends the swab past the distal end of the colposcopic-type viewing extension device. In some embodiments, such colposcopic-type viewing extension device may in fact omit the actual viewing elements of the device to provide an improved, simple way of obtaining a swab of cells from a target tissue. More typically, the swab will be coextensive with the viewing channel(s) of the colposcopic-type viewing extension device.

In certain embodiments, the viewing screen as discussed before can be directly located in-line with the viewing channel(s) or alternatively the viewing screen can be a “flip-up” configuration so that the device is more easily stored and/or manipulated prior to engagement of the viewing screen and then the viewing screen can be more easily seen by the user. The colposcopic-type viewing extension device as discussed herein can also include a plurality of different swab(s), spatula(s), punch biopsy(-ies), etc., as desired. As also desired, the colposcopic-type viewing extension device can comprise one or more rotatable handles, extension levers or other systems, typically mechanically operated, to switch from one swab to another or from a swab to a spatula, etc.

If desired, the colposcopic-type viewing extension device can either comprise or be configured to receive one or more replaceable covers to cover at least the portion of the device that may contact the patient. In certain embodiments, such covers are simply thin plastic covers (which may comprise one or more holes to permit transmission of swabs, spatulas, liquids, etc., as desired). Typically, such plastic sheaths are easily disposable, although such can instead be cleanable (preferably autoclavable or otherwise sterilizable) so that the same sheath can be used repeatedly. Such sheaths can be either flexible or rigid as desired.

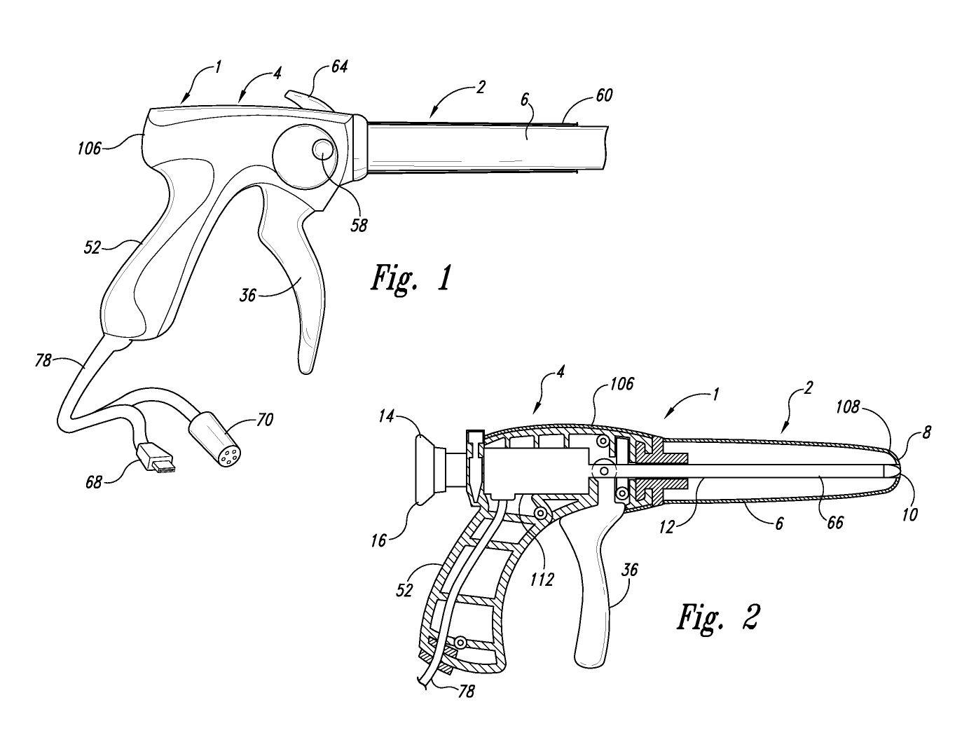

Turning to a discussion of the figures, FIGS. 1 and 2 depict embodiments of a handheld imaging and sampling probe system 1, comprising a probe 2, having a handle 4 and extension 6. As can be seen, the extension 6 extends distally from the handle 4. The handle 4 is sized and configured to be maintained external to a patient while the extension 6 is relatively thin and is of adequate length to reach the cervix of the patient. Further, the extension 6 is sized to be easily and comfortably inserted into a vagina up to the cervix without use of a speculum. (Of course, if desired, a speculum can be used but an advantageous feature of the devices and systems, etc., herein is that a speculum need not be used and therefore the discomfort associated with a speculum need not be suffered by the patient pursuant to use of the probes, etc., discussed herein.) In the embodiment shown in FIG. 2, the system I and probe 2 further comprise a light emitter 8 and an imaging sensor 10 operably coupled to a viewer 14 which in the embodiment in FIG. 2 is an ocular 16.

Returning to FIG. 1, handle 4 comprises a handle body 106 having a trigger 36 configured to extend and retract a swab, spatula, punch biopsy, surgical loop, or other suitable retractable biopsy or surgical device (“RBSD”). In this regard, probe system 1 further comprises an RBSD selector switch 64 as well as an RBSD rotator knob 58. Handle 4 further comprises a grip 52 sized and configured to fit a human hand. In FIG. 1, the probe system is not completely stand-alone, and therefore an external device connector 78 is provided. In the embodiment shown, the external device connector 78 comprises both a computer connector 68 (which in the embodiment shown is a USB connector) as well as an optical fiber connector 70. These connectors or leads connect the probe to one or more external power sources, light sources, computers, viewing screens, spectral analysis equipment, recording devices, sources of liquids or gases such as air, water, or acetic acid (of course, the computer and optical fiber leads shown in FIG. 1 would not transmit gases or fluids, but similar, appropriately structured leads can provide access to such materials). The system 1 in FIG. 1 also depicts, in part, the beginnings of a sheath 60 that covers the extension 2. The sheath 60 can be either disposable or reusable, can be rigid, semi-rigid or pliable, and can be otherwise configured as desired for a given purpose and/or patient.

FIG. 2 provides a cutaway side view of an embodiment of a probe 2 comprising an imaging system 12 including a light emitter 8, an imaging sensor 10, and a viewer 14 in the form of an ocular 16. In particular, probe 2 comprises a direct viewing structure such that a user can directly look through ocular 16 to a target tissue. Light is emanated by a light emitter 8, which can be, for example, a distally located LED or other light generating device, or an appropriate light transmitter, such as a fiber optic guide or a liquid light guide, which conducts light from an external light source. The optical system in FIG. 2 comprises a substantially hollow passage 66 through which the user looks from the proximal end of the probe 2 through to the distal tip 108 of the probe 2. Thus, hollow passage 66 transmits light directly from the target tissue to the eye of the user. Any desirable optics can be included in the viewing system (in this and other embodiments) such as lenses, mirrors, filters, etc. For example, within chamber 112 can be a dichroic mirror (not shown) that reflects a portion of the examination light received from the target tissue down external device connector 78 while transmitting adequate light, typically the significant bulk of the light, through to the ocular 16 so that the eye of the user can readily see the target tissue and properly manipulate the probe 2 to obtain the desired sample or conduct other desired actions related to the target tissue.

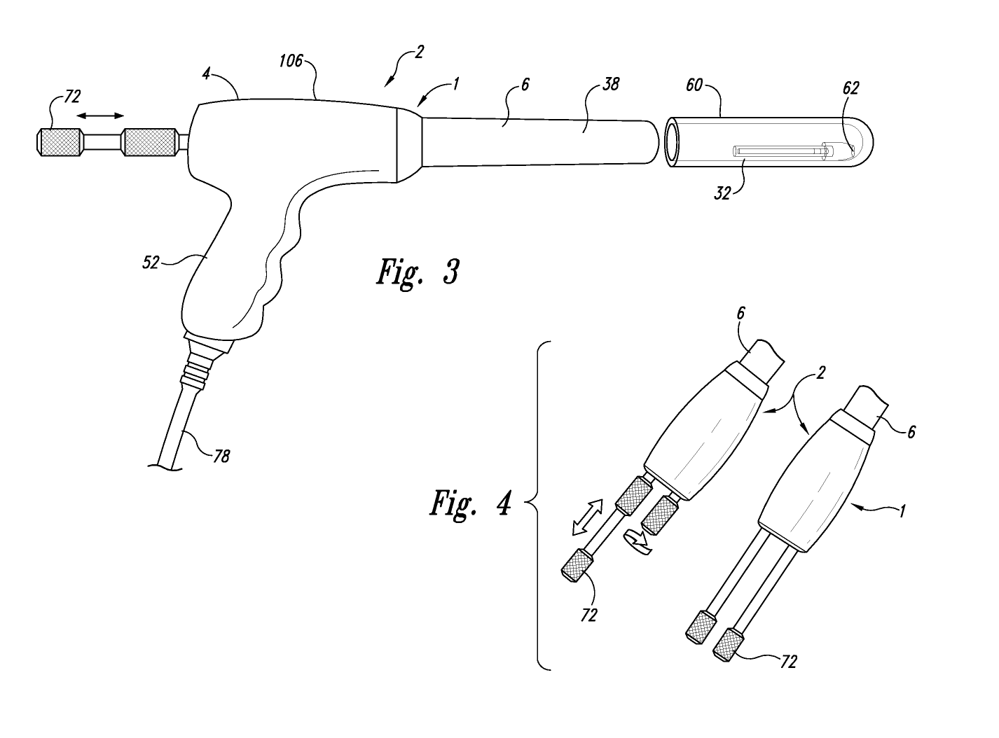

Turning to FIG. 3, the probe 2 in FIG. 3 comprises a handle 4 and an extension 6 as well as a grip 52 and an external device connector 78. In FIG. 3, the extension 6 includes a distal end 38 over which a disposable sheath 60 is placed. Disposable sheath 60 comprises a biopsy channel 32 as well as a port 62 through which the RBSD can pass.

In FIGS. 3 and 4, the RBSD either comprises or can be controlled by a proximally located manual RBSD manipulator 72 which as can be seen can be pushed in, pulled out, and twisted right or left or otherwise manipulated as desired. In the embodiments shown in FIG. 4, two such manual RBSD manipulators 72 are provided, one on either side of the probe 2. Of course, more biopsy channels and/or RBSDs can be provided if desired. Each manual RBSD manipulator 72 can comprise or be operably connected to two of the same RBSDs (e.g., two spatulas) or two different RBSDs, such as a spatula, a swab, a surgical loop, a punch biopsy, a bioptome, etc.

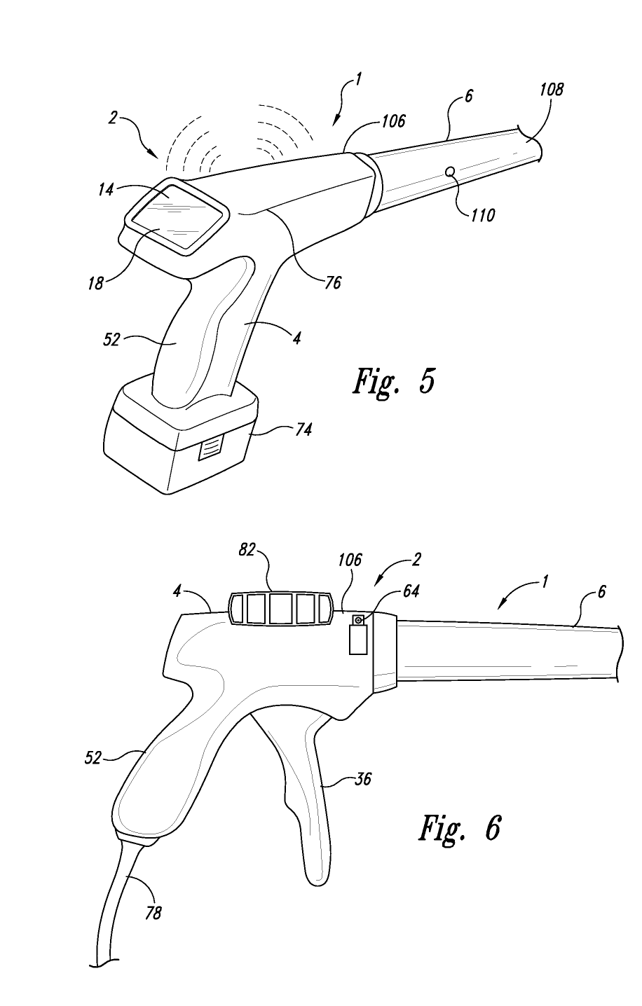

FIG. 5 depicts a completely wireless embodiment of the handheld probe 2. In this embodiment, handle 4 and grip 52 are located above a battery 74. The battery provides electrical power to charge a distally located light source (or light source that could be located within battery pack 74 with light conducted from the light source to the desired location along extension 6), such as side port 110. Such a side port can be advantageous, for example, for examining, sampling, etc., laterally located structures such as the vaginal wall. In FIG. 5, the wireless system also comprises a radio transmitter 76 that can transmit any desired information to a suitable radio receiver, for example imaging information, spectral analysis information, etc. The probe 2 in FIG. 5 also comprises a viewing screen 18 located on the proximal end of probe 2. In the embodiment shown, the viewing screen is substantially directly in line with the distal tip 108 of extension 6 so that manipulation of the probe 2 is intuitive for the user.

In FIG. 6, a probe 2 comprises an RBSD and image sensor manipulator 82 in the form of a knob located on the top surface of the handle 4. The knob can be twisted and/or otherwise manipulated to cause the RBSD to extend, retract, rotate, or otherwise perform as desired.

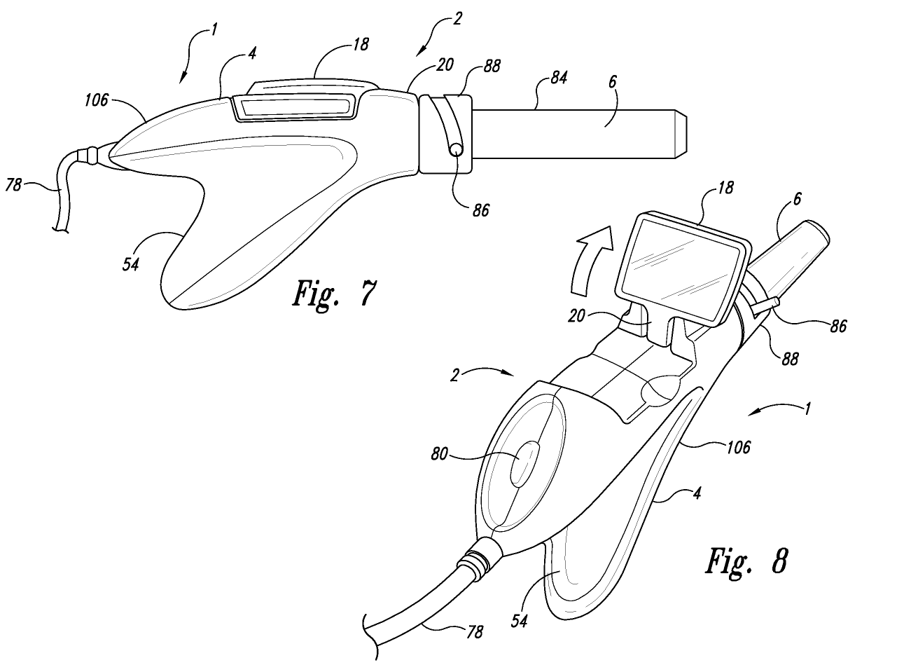

FIGS. 7 and 8 depict an embodiment of a probe 2 comprising a handle 4 having a soft grip 54 in a substantially in-line configuration with extension 6. Probe 2 also comprises a flip-up viewing screen 18 connected via a flip-up connector 20 to handle 4. In FIGS. 7 and 8, extension 6 is a sterilizable extension 84 that is modular and thus can be completely detached then reattached to handle 4. In the embodiment in FIGS. 7 and 8, sterilizable extension 84 is attached to handle 4 at sterilizable extension collar 88 which can be made of any suitable material (like other components herein), for example stainless steel. Once placed within sterilizable extension collar 88, sterilizable extension 84 can be retained in place by pushing sterilizable extension retention knob 86 from one side to the other, thereby locking sterilizable extension 84 in place. FIG. 8 also depicts a power button 80 to provide an easy way to turn system 1 on and off.

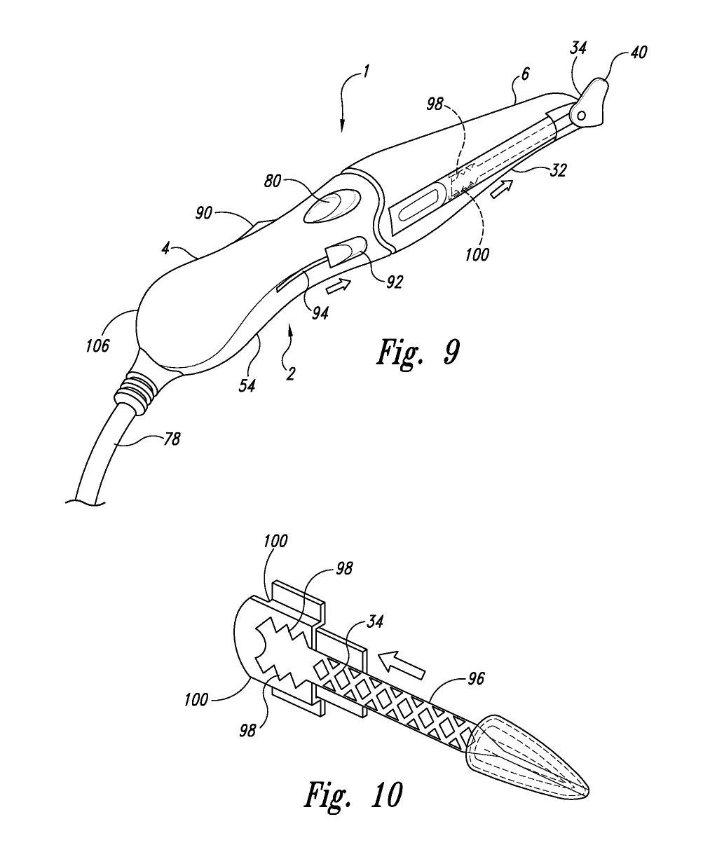

FIG. 9 depicts a further embodiment of an in-line probe 2 having a superior mounted on-off switch 80 and opposed thumb slides 90, 92 for a retractable biopsy or surgical device (RBSD) 34 such as a swab (not shown) and a spatula 40, respectively. The thumb slides 90 and 92 are disposed in respective knob recessions 94. As can be seen, spatula 40 slides back and forth through biopsy channel 32. In this embodiment, rotation of the spatula or swab is achieved by rotating the probe itself instead of holding the probe substantially still and rotating the spatula (or other RBSD) relative to both the probe 2 and the target tissue of the patient. As can be seen in the cutaway close-up FIG. 10, as well as in FIG. 9, in the embodiments shown, the RBSD (i e., spatula 40 in FIG. 9 and broom 96 in FIG. 10) are connected to extension 6 but not handle 4. In FIGS. 9 and 10, the RBSD is connected via a detent 98 and projection 100 system. Any suitable connection system can be employed, typically an easily detachable retention system.

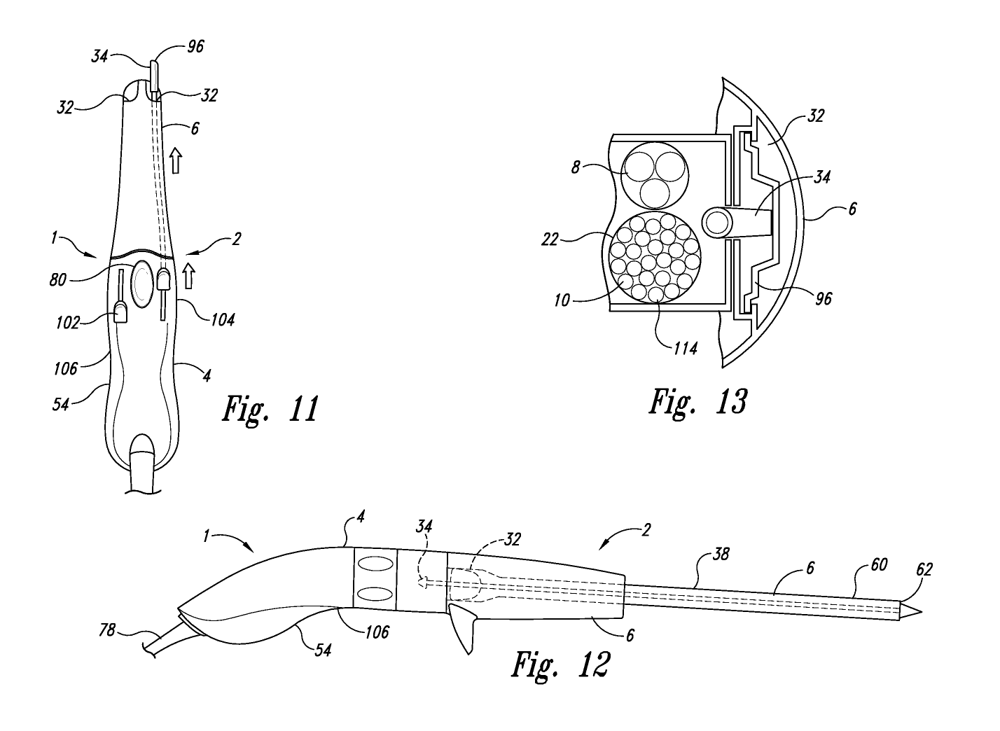

FIG. 11 depicts an embodiment of a device substantially similar to that in FIGS. 9 and 10 in slides 90, 92 have been renumbered as knob for swab extension and retraction 102 and knob for biopsy device extension and retraction 104 to more clearly indicate that in this embodiment each of the knobs are used for extension and retraction, and can be used for different devices and indeed that only one need be deployed at a given time.

FIG. 12 depicts a further embodiment of a probe 2 wherein retractable biopsy or surgical device RBSD 34, wherein the RBSD 34 is transmitted to the target tissue via a biopsy channel 32 extending along a side of the extension 6 and into a distal end 38 of extension 6. Distal end 38 is in turn encompassed within a sheath 60 which sheath can also be configured to also cover distal end 6 all the way to handle 4, and further to cover handle 4 and other structures as desired. In FIG. 12, RBSD 34 is manually threaded biopsy channel 32 through to a port 62 in the sheath 60 where it can then be contacted with the target tissue. As can be seen, RBSD 34 is not in this embodiment attached to any manipulation devices in the probe itself, but rather is simply manually manipulated by the user.

FIG. 13 depicts a cutaway, cross-sectional end view of an extension member 6 comprising a biopsy channel 32, an RBSD 96, and a fiber bundle imaging sensor 22. As can be seen, the fiber bundle imaging sensor 22 comprises a large plurality of individual optical fibers 114 configured to provide a pixelated view of the target tissue to an externally located imaging device such as the flip-up screen in FIGS. 7 and 8, the distally located screen 18 or even a stand-alone imager such as a computer screen, as well as, if desired, direct connection via appropriate optics to an ocular or other suitable viewer such as ground glass (for example, the directly attached viewing screen 18 in FIG. 5 could be either an electronically powered viewing screen or a ground glass viewing screen providing an image of examination light transmitted directly to such viewer 14).

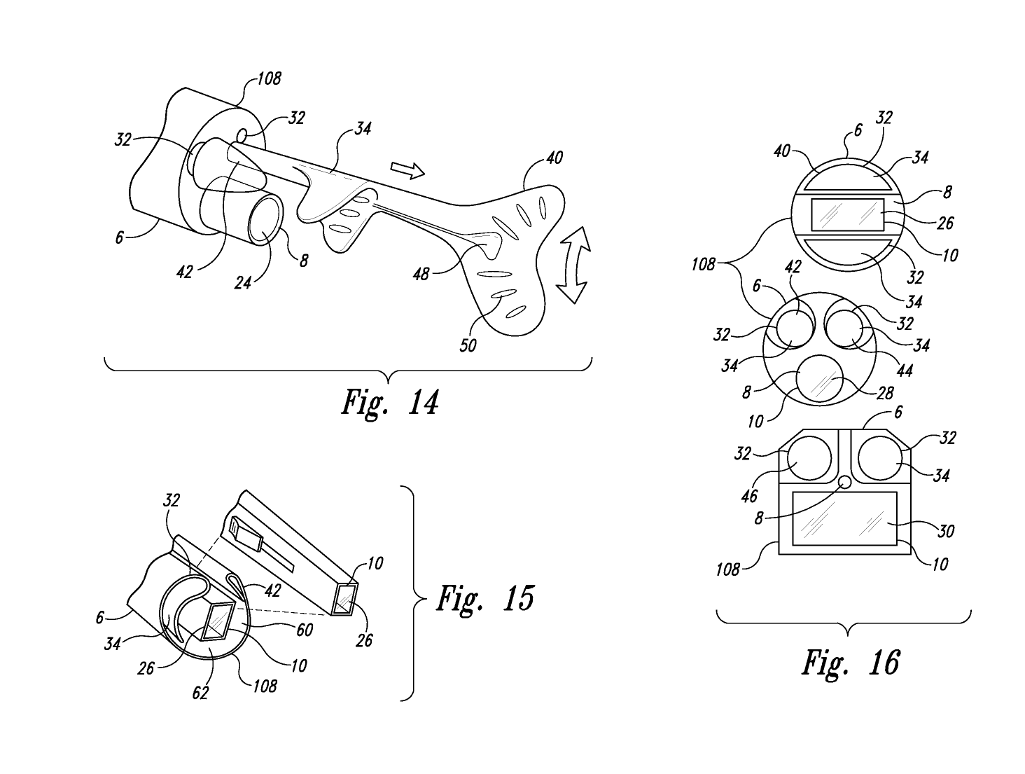

FIGS. 14-16 depict close-up views of an exemplary distal tips 108 of extension 6 comprising multiple biopsy channels 32, multiple RBSDs 34, and, in FIG. 14, a snapshot camera 24. As can be seen, each of the devices are extensible and retractable relative to the distal tip 108. The depiction of spatula 40 in FIG. 14 shows the spatula both in a retracted state wherein the spatula is substantially folded in on itself and in an extended state wherein relief notch 48 and structural ribs 50 in the spatula 40 can be seen. In FIG. 14, the RBSD 34 in the other biopsy channel 32 is a swab 42. FIGS. 15 and 16 depict additional embodiments of exemplary distal tips 108 of probe 2. In FIG. 15, distal tip 108 includes a CCD video camera 26, while the embodiments in FIG. 16 include a round video camera 28, a bioptome 44, and a fluid dispenser 46. The embodiments in FIG. 16 also demonstrate that the distal tip 108 need not be round in cross-section.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

A handheld imaging and sampling probe system wherein the handheld probe can comprise a proximally located handle and at least one relatively thin extension that extends distally from the handle, wherein the handle can be sized and configured to be maintained external to a patient and the extension can be of adequate length to at least substantially reach to a cervix of a patient and can be sized to be easily and comfortably inserted into a vagina up to a cervix without use of a speculum, the probe further can comprise at least one light emitter configured to emit illumination light toward a target tissue within the vagina, at least one imaging system can comprise an imaging sensor configured to detect examination light emanating from the target tissue and a viewer configured to provide an image of the target tissue at the handle, and at least one biopsy channel in at least the extension that can be configured to transmit to the target tissue a retractable biopsy or surgical device able to retractably contact the target tissue. The system further can comprise a removable sleeve sized to substantially cover the extension to be inserted into the vagina, the sleeve can comprise at least one passage configured to securely align with the biopsy channel and sized to transmit the retractable biopsy or surgical device, and the extension can be about 1 cm in diameter.

The imaging system can comprise a substantially hollow tube configured to transmit the examination light from the target tissue to the user to provide direct viewing of the target tissue by the user, the viewer can be at least one of an ocular or a ground glass screen. The imaging system can comprise at least one imaging sensor at the distal end operably coupled to a viewer comprising a viewing screen proximally disposed on the probe; the viewing screen can be a flip-up viewing screen. The imaging sensor can be located along a side of the extension such that the imaging sensor can be configured to view a vaginal wall, and the viewer can be in substantially linear relation to the distal end of the probe such that manipulation of the probe by the user provides an intuitive change of the field of view for the user.

The system further can be a stand-alone viewing device and the images can be transmitted via at least one of a wire or radio frequency or other wireless form of communication from the probe to the stand-alone viewing device. The system further can comprise at least one spectroscopic sensor configured to receive the examination light, such as a spectrometer, spectrophotometer, spectroradiometer, and a spectrograph. The retractable biopsy or surgical device can be extensible and retractable via a loaded spring configured such that the retractable biopsy or surgical device can be extended by pushing on the collection device a first time then retracted by pushing on the collection device a second time whereupon the loaded spring forces the collection device back up the biopsy channel. The probe further can comprise a snapshot camera configured to take snapshots of the target tissue.

The handle can be shaped either as in-line handle or as a pistol-grip handle. The probe can be stand-alone, wireless and cordless, and the probe further can comprise at least one wireless transmitter and at least one battery sufficient to power the probe during use. At least the extension further can comprise at least one gradation or marking configured such that an additional viewing device can easily determine at least one of the location of the probe, the relative position of the probe or the size of a lesion or tumor in the target tissue, and the probe further can comprise at least one channel configured to conduct a desired composition to the target wherein the material can be at least one of air or liquid. The retractable biopsy or surgical device can be operably connected to the handle such that the retractable biopsy or surgical device can be at least one of pulled, pushed or rotated by manipulating at least one of a knob or lever on the handle. When retracted the retractable biopsy or surgical device can be substantially coextensive with the distal tip of the imaging system. The probe can comprise at least a first and second retractable biopsy or surgical devices and the probe further can comprise at least one rotatable knob or lever configured to switch from the first retractable biopsy or surgical device to the second retractable biopsy or surgical device.

In another aspect, a kit can comprise the handheld imaging and sampling probe systems herein along with a disposable sleeve sized to substantially cover the extension to be inserted into the vagina, the sleeve can comprise at least one passage configured to securely align with the biopsy channel and sized to transmit the retractable biopsy or surgical device. The kit further can comprise at least one retractable biopsy or surgical device able to retractably contact the target tissue through the biopsy channel, and instructions approved by a medical regulation body such as the FDA relating to at least one of examination or biopsy of a cervix using the handheld imaging and sampling probe system and at least one of the sleeve and the retractable biopsy or surgical device.

In further aspect, methods of making a handheld imaging and sampling probe system can comprise: joining a handle sized and configured to be maintained external to a patient and at least one relatively thin extension that extends distally from the handle wherein the extension can be of adequate length to at least substantially reach to a cervix of a patient and can be sized to be easily and comfortably inserted into a vagina up to a cervix without use of a speculum, providing in or on the extension at least one light emitter configured to emit illumination light toward a target tissue within the vagina, providing in or on the probe at least one imaging system can comprise an imaging sensor configured to detect examination light emanating from the target tissue and a viewer configured to provide an image of the target tissue at the handle, and making at least one biopsy channel in at least the extension, the biopsy channel configured to transmit to the target tissue a retractable biopsy or surgical device able to retractably contact the target tissue.

The methods further can comprise placing over at least a distal end of the extension a disposable sleeve can comprise at least one passage configured to securely align with the biopsy channel and sized to transmit the retractable biopsy or surgical device such that the passage securely align with the biopsy channel. The methods further can comprise retractably transmitting at least one retractable biopsy or surgical device through the biopsy channel.

In still a further aspect, methods of at least one examining or biopsying a structure within a vagina, can comprise: placing a handheld imaging and sampling probe system according to claim in the vagina without use of a speculum and without discomfort to the patient, and examining or biopsying a structure within the vagina. The methods further can comprise placing over at least a distal end of the extension a disposable sleeve can comprise at least one passage configured to securely align with the biopsy channel and sized to transmit the retractable biopsy or surgical device such that the passage securely align with the biopsy channel. The methods further can comprise examining the target tissue via the imaging system, retractably transmitting at least one retractable biopsy or surgical device through the biopsy channel, biopsying the target tissue, spectrally analyzing the target tissue, and/or swabbing the target tissue without retracting the handheld imaging and sampling probe method from the patient.

Unless expressly indicated otherwise, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

1. A handheld imaging and sampling probe system, the handheld probe comprising a proximally located handle and at least one relatively thin extension that extends distally from the handle, wherein the handle is sized and configured to be maintained external to a patient and the extension is of adequate length to at least substantially reach to a cervix of a patient and is sized to be easily and comfortably inserted into a vagina up to a cervix without use of a speculum, the probe further comprising at least one light emitter configured to emit illumination light toward a target tissue within the vagina, at least one imaging system comprising an imaging sensor configured to detect examination light emanating from the target tissue and a viewer configured to provide an image of the target tissue at the handle, and at least one biopsy channel in at least the extension that is configured to transmit to the target tissue a retractable biopsy or surgical device able to retractably contact the target tissue.

2. The system of claim 1 wherein the system further comprises a removable sleeve sized to substantially cover the extension to be inserted into the vagina, the sleeve comprising at least one passage configured to securely align with the biopsy channel and sized to transmit the retractable biopsy or surgical device.

3. The system of any one of claims 1 to 2 wherein the extension is about 1 cm in diameter.

4. The system of any one of claims 1 to 2 wherein the imaging system comprises a substantially hollow tube configured to transmit the examination light from the target tissue to the user to provide direct viewing of the target tissue by the user.

5. The system of claim 4 wherein the viewer is at least one of an ocular or a ground glass screen.

6. The system of any one of claims 1 to 2 wherein the imaging system comprises at least one imaging sensor at the distal end operably coupled to a viewer comprising a viewing screen proximally disposed on the probe.

7. The system of claim 6 wherein the viewing screen is a flip-up viewing screen.

8. The system of claim 6 wherein the imaging sensor is located along a side of the extension such that the imaging sensor is configured to view a vaginal wall.

9. The system of any one of claims 1 to 2 wherein the viewer is in substantially linear relation to the distal end of the probe such that manipulation of the probe by the user provides an intuitive change of the field of view for the user.

10. The system of any one of claims 1 to 2 wherein the system further comprises a stand-alone viewing device and the images are transmitted via at least one of a wire or radio frequency from the probe to the stand-alone viewing device.

11. The system of any one of claims 1 to 2 wherein the system further comprises at least one spectroscopic sensor configured to receive the examination light.

12. The system of claim 11 wherein the spectroscopic sensor is at least one of a spectrometer, spectrophotometer, spectroradiometer, and a spectrograph.

13. The system of any one of claims 1 to 2 wherein the retractable biopsy or surgical device is extensible and retractable via a loaded spring configured such that the retractable biopsy or surgical device is extended by pushing on the collection device a first time then retracted by pushing on the collection device a second time whereupon the loaded spring forces the collection device back up the biopsy channel.

14. The system of any one of claims 1 to 2 wherein the probe further comprises a snapshot camera configured to take snapshots of the target tissue.

15. The system of any one of claims 1 to 2 wherein the handle is shaped either as in-line handle or as a pistol-grip handle.

16. The system of any one of claims 1 to 2 wherein the probe is stand-alone, wireless and cordless, and wherein the probe further comprises at least one wireless transmitter and at least one battery sufficient to power the probe during use.

17. The system of any one of claims 1 to 2 wherein at least the extension further comprises at least one gradation or marking configured such that an additional viewing device can easily determine at least one of the location of the probe, the relative position of the probe or the size of a lesion or tumor in the target tissue.

18. The system of any one of claims 1 to 2 wherein the probe further comprises at least one channel configured to conduct a desired composition to the target wherein the material is at least one of air or liquid.

19. The system of any one of claims 1 to 2 wherein the retractable biopsy or surgical device is operably connected to the handle such that the retractable biopsy or surgical device can be at least one of pulled, pushed or rotated by manipulating at least one of a knob or lever on the handle.

20. The system of any one of claims 1 to 2 wherein when retracted the retractable biopsy or surgical device is substantially coextensive with the distal tip of the imaging system.

21. The system of any one of claims 1 to 2 wherein the probe comprises at least a first and second retractable biopsy or surgical devices and the probe further comprises at least one rotatable knob or lever configured to switch from the first retractable biopsy or surgical device to the second retractable biopsy or surgical device.

22. A kit comprising the handheld imaging and sampling probe system of claim 1 and a disposable sleeve sized to substantially cover the extension to be inserted into the vagina, the sleeve comprising at least one passage configured to securely align with the biopsy channel and sized to transmit the retractable biopsy or surgical device.

23. The kit of claim 22 further comprising at least one retractable biopsy or surgical device able to retractably contact the target tissue through the biopsy channel.

24. The kit of claim 22 or 23 further comprising instructions approved by a medical regulation body relating to at least one of examination or biopsy of a cervix using the handheld imaging and sampling probe system and at least one of the sleeve and the retractable biopsy or surgical device.

25. A method of making a handheld imaging and sampling probe system comprising:
joining a handle sized and configured to be maintained external to a patient and at least one relatively thin extension that extends distally from the handle wherein the extension is of adequate length to at least substantially reach to a cervix of a patient and is sized to be easily and comfortably inserted into a vagina up to a cervix without use of a speculum,
providing in or on the extension at least one light emitter configured to emit illumination light toward a target tissue within the vagina,
providing in or on the probe at least one imaging system comprising an imaging sensor configured to detect examination light emanating from the target tissue and a viewer configured to provide an image of the target tissue at the handle, and
making at least one biopsy channel in at least the extension, the biopsy channel configured to transmit to the target tissue a retractable biopsy or surgical device able to retractably contact the target tissue.

26. The method of claim 25 wherein the method further comprises placing over at least a distal end of the extension a disposable sleeve comprising at least one passage configured to securely align with the biopsy channel and sized to transmit the retractable biopsy or surgical device such that the passage securely align with the biopsy channel.

27. The method of claim 25 or 26 wherein the method further comprises retractably transmitting at least one retractable biopsy or surgical device through the biopsy channel.

28. A method of at least one examining or biopsying a structure within a vagina, the method comprising:
placing a handheld imaging and sampling probe system according to claim 1 in the vagina without use of a speculum and without discomfort to the patient, and
examining or biopsying a structure within the vagina.

29. The method of claim 28 wherein the method further comprises placing over at least a distal end of the extension a disposable sleeve comprising at least one passage configured to securely align with the biopsy channel and sized to transmit the retractable biopsy or surgical device such that the passage securely align with the biopsy channel.

30. The method of claim 28 or 29 wherein the method further comprises examining the target tissue via the imaging system.

31. The method of any one of claims 28 to 29 wherein the method further comprises retractably transmitting at least one retractable biopsy or surgical device through the biopsy channel.

32. The method of any one of claims 28 to 29 wherein the method further comprises biopsying the target tissue.

33. The method of any one of claims 28 to 29 wherein the method further comprises taking a spectral analysis of the target tissue.

34. The method of any one of claims 28 to 29 wherein the method further comprises both swabbing and biopsying the target tissue without retracting the handheld imaging and sampling probe method from the patient.

35. The method of any one of claims 28 to 29 wherein the target tissue is a cervix.
